(19) United States
(12) Patent Application Publication
(10) Pub. No.: US 2015/0099128 A1
(43) Pub. Date: Apr. 9, 2015

(54) LINEAR GLYCIDYL CARBAMATE (GC) RESINS FOR HIGHLY FLEXIBLE COATINGS

(71) Applicant: NDSU RESEARCH FOUNDATION, Fargo, ND (US)

(72) Inventors: Umesh D. Harkal, Sterling Heights, MI (US); Andrew J. Muehlberg, Fargo, ND (US); Dean C. Webster, Fargo, ND (US)

(21) Appl. No.: 14/394,795

(22) PCT Filed: Apr. 18, 2013
(86) PCT No.: PCT/US13/37118

§ 371 Date: Oct. 16, 2014

Related U.S. Application Data
(60) Provisional application No. 61/635,049, filed on Apr. 18, 2012.

(51) Int. Cl. C09D 175/04; C08G 18/12; C08G 18/73; C07C 269/02; C07C 271/20; C08G 18/28

(52) U.S. Cl. CPC C09D 175/04; C07C 271/20; C08G 18/2845; C08G 18/73; C07C 269/02; C08G 18/12
USPC 428/423.1; 560/158; 528/59; 524/590; 427/386

(57) ABSTRACT

This invention relates to coating compositions comprising a linear glycidyl carbamate (GC) resin and a curing agent. The linear GC-resins were synthesized using linear and cycloaliphatic diisocyanates and a combination of diols and optional triols with glycidol. The combination of linear diisocyanates and diols introduces a more linear structure in the GC-resin compositions.

### CROSS-REFERENCE TO RELATED APPLICATIONS

This application claims priority under 35 U.S.C. §119 to U.S. Provisional Application No. 61/635,049, filed Apr. 18, 2012, which is incorporated herein by reference.

### STATEMENT OF GOVERNMENT RIGHTS

This invention was made with government support under Contract No FA9550-09-C-0150 awarded by the United States Air Force. The government has certain rights in the invention.

### FIELD OF THE INVENTION

This invention relates to a novel glycidyl carbamate coating composition having improved flexibility. Such coating composition comprises a linear glycidyl carbamate resin and a curing agent. The linear glycidyl carbamate resin of the invention comprises the reaction product of an isocyanate terminated urethane compound and glycidol, wherein the isocyanate terminated urethane compound comprises the reaction product of at least one diisocyanate compound, at least one diol compound, and, optionally, at least one triol compound.

### BACKGROUND OF THE INVENTION

Glycidyl carbamate (GC) resins are obtained by the reaction of isocyanate functional compounds with glycidol. A unique property of GC resins is the combination of both urethane and epoxy functional groups in their structures and thus the performance of urethane and reactivity of epoxide are combined in a single resin. The recent primary research on GC resins and coatings was based on resins obtained from aliphatic polyisocyanates such as the biuret and isocyanurate resins of hexamethylene diisocyanate. For example, biuret glycidyl carbamate (BGC) and isocyanurate glycidyl carbamate (IGC) resins were obtained by reacting biuret and isocyanurate polyisocyanate resin with glycidol, respectively. Coatings prepared from BGC and IGC using amine and self-crosslinking had an excellent combination of chemical and mechanical performance properties. See, e.g., Edwards, P A, Striemer, G, Webster, D C, “Novel Polyurethane Technology Through Glycidyl Carbamate Chemistry,” J. Coat. Technol. Res. 2(7):517-527 (2005); Edwards, P A, Striemer, G, Webster, D C, “Synthesis, Characterization and Self-crosslinking of Glycidyl Carbamate Functional Resins,” Prog. Org. Coat. 57(2):128-139 (2006). High performance organic-inorganic hybrid GC coatings can be obtained by sol-gel crosslinking through alkoxy silane groups in the coating network. See, e.g., Chattopadhyay, D K, Muehlberg, A J, Webster, D C, “Organic-inorganic hybrid coatings prepared from glycidyl carbamate resins and amino-functional silanes,” Prog. Org. Coat. 63(4):405-415 (2008); Chattopadhyay, D K, Webster, D C, “Hybrid coatings from novel silane-modified glycidyl carbamate resins and amine crosslinkers,” Prog. Org. Coat. 66(1):73-85 (2009); Chattopadhyay, D K, Zakula, A D, Webster, D C, “Organic-inorganic hybrid coatings prepared from glycidyl carbamate resin, 3-aminopropyl trimethoxy silane and tetraethoxyorthosilicate,” Prog. Org. Coat. 64(2-3):128-137 (2009). Additionally, it was shown that lower viscosity modified GC resins and hydrophilically modified water dispersible GC resins can be obtained for low VOC applications. See, e.g., Harkal, U D, Muehlberg, A J, Li, J, Garrett, J T, Webster, D C, “The influence of structural modification and composition of glycidyl carbamate resins on their viscosity and coating performance,” J. Coat. Technol. Res. 7(5):531-546 (2010); Harkal, U D, Muehlberg, A J, Edwards, P A, Webster, D C, “Novel water-dispersible glycidyl carbamate (GC) resins and waterborne amine-cured coatings,” J. Coat. Technol. Res. 8(6):735-747 (2011).

The reaction of an isocyanate functional compound with an alcohol produces urethane functionality. The formation of reversible hydrogen bonding between urethane groups improves scratch resistance and toughness. Polyurethane coatings with a diverse range of properties are obtained by varying the composition of diisocyanates and polyisocyanates with diols and polyols. The fundamental chemical nature and molecular architecture of isocyanates and alcohols used to obtain urethanes can have a profound influence on the crosslinked network and coating properties such as mechanical properties (elongation at break, modulus, scratch resistance, hardness, adhesion, etc), glass transition temperature (T_{g}), thermal stability, and resistance to chemicals, corrosion, and weathering. For example, polyurethanes based on symmetric diisocyanates such as hexamethylene diisocyanate (HDI), 1,4-phenylene diisocyanate, or 1,4-cyclohexyl diisocyanate result in flexible polymer films. High solids polyurethane coatings based on symmetric diisocyanates and linear diols show high elongation at break. Polyurethane coatings based on bis(4-isocyanatocyclohexyl)methane (H₁₂MDI), and isophorone diisocyanate (IPDI) exhibit high strength, stiffness, and hardness. See, e.g., Wingborg, N, “Increasing the tensile strength of HTPB with different isocyanates and chain extenders,” Polym. Test. 21(3):283-287 (2002); Ni, H, Daum, J L, Soucek, M D, Simonsick, W J, Jr., “Cycloaliphatic polyester based high solids polyurethane coatings: I. The effect of difunctional alcohols,” J. Coat. Technol. 74(928):49-56 (2002); Yilgor, I, Yilgor, E, “Structure-Morphology-Property Behavior of Segmented Thermoplastic Polyurethanes and Polyureas Prepared without Chain Extenders,” Polym. Rev. (Philadelphia, Pa., U.S.) 47(4):487-510 (2007); Dearth, R S, Mertes, H, Jacobs, P J, “An overview of the structure/property relationship of coatings based on 4,4′-dicyclohexylmethane diisocyanate (H12MDI),” Prog. Org. Coat. 29(1-4):73-79 (1996); Yoo, H-J, Lee, Y-H, Kwon, J-Y, Kim, H-D, “Comparison of the properties of UV-cured polyurethane acrylates containing different diisocyanates and low molecular weight diols,” Fibers Polym. 2(3):122-128 (2001).

Highly flexible coatings are needed in many industries such as electronics, packaging, automotive, and aviation. See, e.g., Lange, J, Stenroos, E, Johansson, M, Malmstrom, E, “Barrier coatings for flexible packaging based on hyperbranched resins,” Polymer 42(17):7403-7410 (2001); Choi, M-C, Kim, Y, Ha, C-S, “Polymers for flexible displays: From material selection to device applications,” Prog. Polym. Sci. 33(6):581-630 (2008). For example, coating systems, due to lack of flexibility, are prone to fail around joints and riveted parts. See, e.g., Wicks (Jr), Z W, Jones, F N, Pappas, S P, Wicks, D A, Organic Coatings: Science and Technology, 3^{rd} ed., John Wiley and Sons Inc., New Jersey (2007); Baboion, R, Corrosion Tests and Standards Applications and Interpretations, 2^{nd} ed., ASTM International, West Conshohocken (2005). In some applications, coating flexibility can increase the corrosion resistance of coatings by improving barrier properties and protecting the substrate from corrosion. Damage to coatings exposes the metal surface to the corrosive environment and initiates electrochemical corrosion reactions. At this point, corrosion inhibitors are relied upon to slow down the rate of corrosion reactions.

There have been many efforts to develop highly flexible coating systems with good corrosion performance for aircraft applications. Traditional aircraft coatings are based on epoxy-polyamide primer systems, or epoxy-polysulfide rubbers with a flexible polyurethane top coat. See, e.g., Wicks (Jr), Z W, Jones, F N, Pappas, S P, Wicks, D A, Organic Coatings: Science and Technology, 3^{rd} ed., John Wiley and Sons Inc., New Jersey (2007); Bierwagen, G, Brown, R, Battocchi, D, Hayes, S, “Active metal-based corrosion protective coating systems for aircraft requiring no-chromate pretreatment,” Prog. Org. Coat. 68(1-2):48-61 (2010); U.S. Pat. No. 4,720,405; U.S. Pat. No. 4,680,346; U.S. Pat. No. 4,101,497; U.S. Pat. App. Pub. No. 2005/0288456 A1; U.S. Pat. No. 4,692,382; Bierwagen, G, “Next generation of aircraft coatings systems,” J. Coat. Technol. 73(915):45-52 (2001).

This invention relates to the development of highly flexible amine-cured GC-based coatings by designing GC functional resins having structures that are more linear than reported before. According to the invention, linear aliphatic diisocyanates were used in combination with mainly linear diols and glycidol to obtain several GC resins and their amine crosslinked coatings. The coating systems of the invention were shown to have superior flexibility and solvent resistance using reverse impact and MEK double rubs tests. The flexibility of selected coatings was further demonstrated by obtaining values for elongation at break in tensile tests. Differential scanning calorimetry (DSC), dynamic mechanical analysis (DMA), and thermo gravimetric analysis (TGA) on the selected coatings were performed to further understand the structure-property correlations. Corrosion resistance of the selected coatings was demonstrated using salt spray tests.

### SUMMARY OF THE INVENTION

In one embodiment, the invention relates to an isocyanate terminated urethane compound comprising the reaction product of at least one diisocyanate compound, at least one diol compound, and, optionally, at least one triol compound.

In another embodiment, the invention relates to a linear GC-resin comprising the reaction product of an isocyanate terminated urethane compound and glycidol, wherein the isocyanate terminated urethane compound comprises the reaction product of at least one diisocyanate compound, at least one diol compound, and, optionally, at least one triol compound.

In another embodiment, the invention relates to a coating composition comprising at least one linear GC-resin of the invention and at least one curing agent, preferably an amine curing agent.

In another embodiment, the invention relates to a method of making an isocyanate terminated urethane compound of the invention. In one embodiment, this method comprises reacting at least one diisocyanate compound, at least one diol compound, and, optionally, at least one triol compound to make an isocyanate terminated urethane compound of the invention.

In another embodiment, the invention relates to a method of making a linear GC-resin of the invention. In one embodiment, this method comprises reacting an isocyanate terminated urethane compound of the invention with glycidol to make the linear GC-resins of the invention.

In another embodiment, the invention relates to a method of making a coating composition of the invention. In one embodiment, this method comprises curing at least one linear GC-resin of the invention with at least one curing agent, preferably an amine-curing agent.

In another embodiment, the invention relates to an article of manufacture comprising a coating composition of the invention and a method of making such article.

Other features, objects, and advantages of the invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art.

### BRIEF DESCRIPTION OF THE FIGURES

FIG. 1 shows structures of exemplary diisocyanates, diols, triol, and glycidol used in the synthesis of the linear GC-resins.

FIG. 2 shows stress vs. strain plots for coatings F1, F2, F3, L1, and L2.

FIG. 3 shows (a) storage modulus and (b) tan δ curves for coatings F1, F2, F3, L1, L2, and L3.

FIG. 4 shows TGA plots of coatings F1, F2, F3, L1, L2, and L3.

FIG. 5 shows images of coatings L1, L2, and L3 coatings on steel and aluminum substrates after 240 hrs of salt spray.

### DESCRIPTION OF THE INVENTION

### Terminology and Definitions

Unless otherwise indicated, the invention is not limited to specific reactants, substituents, catalysts, catalyst compositions, resin compositions, reaction conditions, or the like, as such may vary. It is also to be understood that the terminology used herein is for the purpose of describing particular embodiments only and is not to be interpreted as being limiting.

As used in the specification and the appended claims, the singular forms “a,” “an,” and “the” include plural referents unless the context clearly dictates otherwise. Thus, for example, reference to “a diisocyanate” includes a single diisocyanate as well as a combination or mixture of two or more diisocyanates, reference to “a diol” encompasses a single diol as well as two or more diols, and the like.

As used in the specification and the appended claims, the terms “for example,” “for instance,” “such as,” or “including” are meant to introduce examples that further clarify more general subject matter. Unless otherwise specified, these examples are provided only as an aid for understanding the invention, and are not meant to be limiting in any fashion.

In this specification and in the claims that follow, reference will be made to a number of terms, which shall be defined to have the following meanings:

The term “alkyl” means a straight or branched saturated hydrocarbyl chains. Non-limiting examples of alkyl groups include methyl, ethyl, n-propyl, isopropyl, n-butyl, isobutyl, sec-butyl, t-butyl, pentyl, iso-amyl, and hexyl. The term “alkylene” denotes a divalent saturated hydrocarbyl chain which may be linear or branched. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkenyl” means a straight or branched hydrocarbyl chain containing one or more double bonds. Each carbon-carbon double bond may have either cis or trans geometry within the alkenyl moiety, relative to groups substituted on the double bond carbons. Non-limiting examples of alkenyl groups include ethenyl (vinyl), 2-propenyl, 3-propenyl, 1,4-pentadienyl, 1,4-butadienyl, 1-butenyl, 2-butenyl, and 3-butenyl. The term “alkenylene” refers to a divalent unsaturated hydrocarbyl chain which may be linear or branched and which has at least one carbon-carbon double bond. Non-limiting examples of alkenylene groups include —C(H)═C(H)— —C(H)═C(H)—CH₂—, —C(H)═C(H)—CH₂—CH₂—, —CH₂—C(H)═C(H)—CH₂—, —C(H)═C(H)—CH(CH₃)—, and —CH₂—C(H)═C(H)—CH(CH₂CH₃)—.

The term “alkynyl” means a straight or branched hydrocarbyl chain containing one or more triple bonds. Non-limiting examples of alkynyl include ethynyl, 1-propynyl, 2-propynyl, 3-propynyl, decynyl, 1-butynyl, 2-butynyl, and 3-butynyl. The term “alkynylene” refers to a divalent unsaturated hydrocarbon group which may be linear or branched and which has at least one carbon-carbon triple bonds. Representative alkynylene groups include, by way of example, —OC—, —OC—CH₂—, —OC—CH₂—CH₂—, —CH₂—OC—CH₂—, —OC—CH(CH₃)—, and —CH₂—C═C—CH(CH₂CH₃)—.

The term “cycloalkyl” refers to a saturated carbocycle group containing zero heteroatom ring members and containing three to ten, preferably three to seven carbon atoms. Non-limiting examples of cycloalkyls include cyclopropyl, cyclobutyl, cyclopentyl, cyclohexyl, cycloheptyl, cyclooctyl, decalinyl, and norpinanyl.

The term “alkoxy” as used herein refers to an alkyl group bound through a single, terminal ether linkage; that is, an “alkoxy” group may be represented as —O-alkyl where alkyl is as defined above.

The terms “halo” and “halogen” are used in the conventional sense to refer to a chloro, bromo, fluoro, or iodo substituent.

“Hydrocarbyl” refers to univalent hydrocarbyl radicals containing 1 to about 30 carbon atoms, preferably 1 to about 24 carbon atoms, most preferably 1 to about 12 carbon atoms, including linear, branched, cyclic, saturated, and unsaturated species, such as alkyl groups, alkenyl groups, alkynyl groups, and the like.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

### Isocyanate Terminated Urethane Compound

The present invention relates to isocyanate terminated urethane compounds comprising the reaction product of at least one diisocyanate compound, at least one diol compound, and, optionally, at least one triol compound.

The diisocyanate compound has the following structure:

O═C═N—R—N═C═O

The diisocyanate is not limited in the divalent group R linking the two isocyanates in the molecule. R is selected from a divalent hydrocarbyl group, including, for example, aliphatic and cyclic structures. For example, R may be a straight or branched C₂-C₁₈ alkylene group, C₂-C₁₈ alkenylene group, or C₂-C₁₈ alkynylene group. R may also be a divalent, cyclic group such as cyclopentyl, cyclohexyl, phenyl, etc. The cyclic group may be saturated or unsaturated, aromatic or non-aromatic, may optionally contain at least one heteroatom or have substituents off a ring atom. R may also be a divalent group having a combination of aliphatic and cyclic structures.

Preferably, R is independently an optionally substituted divalent C₁-C₁₅ alkyl (e.g., hexamethylene), optionally substituted divalent C₃-C₁₅ cycloalkyl, or a divalent substituent selected from the group consisting of:

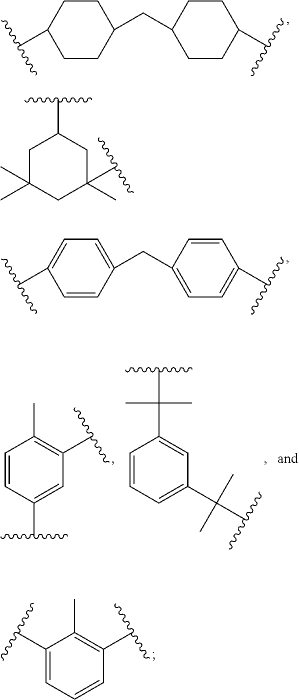

Exemplary diisocyanates that may be used in the invention include, but are not limited to, hexamethylene diisocyanate (HDI), trimethyl hexamethylene diisocyanate (TMDI), dicyclohexyl diisocyanate (H₁₂MDI), isophorone diisocyanate (IPDI), 4,4′-methylene diphenyl diisocyanate (4,4′-MDI), 2,2′-methylene diphenyl diisocyanate (2,2′-MDI), 2,4′-methylene diphenyl diisocyanate (2,4′-MDI), 1,3-phenylene diisocyanate, 1,4-phenylene diisocyanate, 1,4-cyclohexyl diisocyanate, meta-tetramethylxylylene diisocyanate (meta-TMXDI), 2,4-toluene diisocyanate (TDI), 2,6-toluene diisocyanate (TDI), and mixtures thereof.

The diol compound has the following structure:

HO—R′—OH

The diol is not limited by the divalent group R′ linking the hydroxyl groups in the molecule. R′, just as with R, may be selected from a divalent hydrocarbyl group, including, for example, aliphatic and cyclic structures. In addition, R′ may be a divalent ether group, such as, for example, di(C₂-C₅alkylene)ether. R′ may be substituted with any number of substituents or functional moieties. Examples of substituents include, but are not limited to, halo substituents, e.g., F, Cl, Br, or I; a C₁-C₆ alkoxy group, e.g., —OCH₃, —OCH₂CH₃, or —OCH(CH₃)₂; a C₁-C₆ haloalkyl group, e.g., —CF₃, —CH₂CF₃, or —CHCl₂; C₁-C₆ alkylthio; amino; mono and dialkyl amino groups; —NO₂; —CN; a sulfate group, and the like.

In one embodiment, diols that may be used in the invention include, but are not limited to, C₂-C₁₀ alkyl diols and C₂-C₁₀ alkylether diols. For example, exemplary diols that may be used in the invention include, but are not limited to, diethyleneglycol (DEG), 2-butyl-2-ethyl-1,3-propane diol (BEPD), ethylene glycol, 1,2-propane diol, 1,3-propane diol, 2-methyl-1,3-propane diol, 1,4-butanediol, 2,3-butanediol, 1,5-pentanediol, 1,6-hexanediol, 1,4-cyclohexanedimethanol, neopentyl glycol (NPG), and mixtures thereof.

The optional triol compound that may be used in the invention includes, but is not limited to, C₃-C₁₀ alkyl triols. For example, exemplary triols that may be used in the invention include, but are not limited to, trimethylolpropane (TMP), trimethylol ethane (TME), glycerol, and mixtures thereof. Triols may be added to introduce some branched oligomers, in addition to the linear GC-resins of the invention, described below.

The isocyanate terminated urethane compounds can be prepared by a variety of methods. In one embodiment, this method comprises reacting at least one diisocyanate compound, at least one diol compound, and, optionally, at least one triol compound to make an isocyanate terminated urethane compound of the invention. As a non-limiting example, the isocyanate terminated urethane compounds can be prepared by combining at least one diisocyanate compound, at least one diol compound, and, optionally, at least one triol compound in the presence of at least one optional solvent, such as t-butyl acetate (TBA), n-butyl acetate (BA), acetone, methyl ethyl ketone (MEK), methyl n-amyl ketone (MAK), toluene, xylene, ethyl 3-ethoxyproprionate (EEP), and at least one optional catalyst, such as dibutyltindilaurate (DBTDL). In one embodiment, the at least one diol and at least one optional triol may first be heated to melt and mixed with the at least one optional solvent before addition of the diisocyanate and at least one optional solvent. The at least one optional catalyst may then be added after the completion of mixing.

In one embodiment, a stoichiometric excess of the diisocyanate compound is used relative to the at least one diol compound and the optional at least one triol compound. For example, 3 mols of the diisocyanate compound may be combined with 2 mols of the at least one diol to yield an isocyanate terminated urethane compound of the invention having an average of 5 monomer units, as shown in Scheme I below.

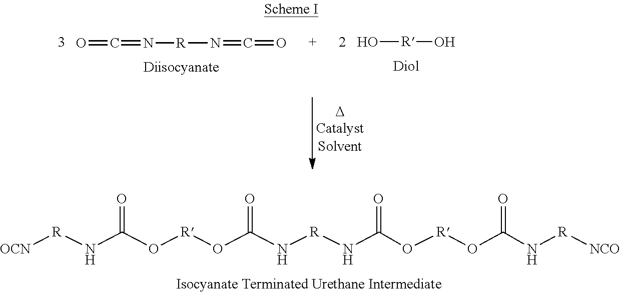

The molar ratio of isocyanate and hydroxyl groups used for the synthesis of the isocyanate terminated urethane compound may range from 1.0:0.66 to 1.0:0.99, more preferably 1.0:0.66 to 1.0:0.75. The amount of triol used depends upon the degree of branched oligomers desired. The molar ratio of diol:triol may range from 1.0:0.05 to 1.0:0.9, more preferably from 1.0:0.05 to 1.0:0.2. Then, as described below, the unreacted isocyanate groups may be reacted with glycidol to yield the final linear GC-resin.

In one embodiment, the solvent may be present in an amount ranging from about 0.1% to about 50.0% by wt., preferably about 0.5% to about 15.0% by wt., even more preferably about 1.0% to about 2.0% by wt., of the total reaction mixture. Solvents may be used during the synthesis to reduce viscosity and facilitate the synthesis reaction.

In one embodiment, the catalyst may be present in an amount ranging from about 0.01% to about 0.1% by wt., more preferably about 0.01% to about 0.05% by wt., of the total reaction mixture.

In one embodiment, the reaction to make the isocyanate terminated urethane compound of the invention may be carried out from about 40° C. to about 90° C., more preferably from about 65° C. to about 80° C. The reaction temperature may be adjusted in order to reach the required value for % NCO (determined by titration) in the isocyanate terminated urethane compound.

### Linear Glycidyl Carbamate Resin

The present invention also relates to linear GC-resins comprising the reaction product of an isocyanate terminated urethane compound and glycidol, wherein the isocyanate terminated urethane compound comprises the reaction product of at least one diisocyanate compound, at least one diol compound, and, optionally, at least one triol compound.

The linear GC-resins can be prepared by a variety of methods. In one embodiment, this method comprises reacting an isocyanate terminated urethane compound of the invention with glycidol to make the linear GC-resins of the invention. As a non-limiting example, the linear GC-resins can be prepared by combining an isocyanate terminated urethane compound, described above, and glycidol in the presence of at least one optional solvent, such as t-butyl acetate (TBA), methyl n-amyl ketone (MAK), ethyl 3-ethoxyproprionate (EEP), and at least one optional catalyst, such as dibutyltindilaurate (DBTDL). The type and amount of solvent and catalyst used to make the linear GC-resin may be the same or different as the type and amount of solvent and catalyst used to make the isocyanate terminated urethane compound described above. Scheme II shows an exemplary synthesis of the linear GC-resins.

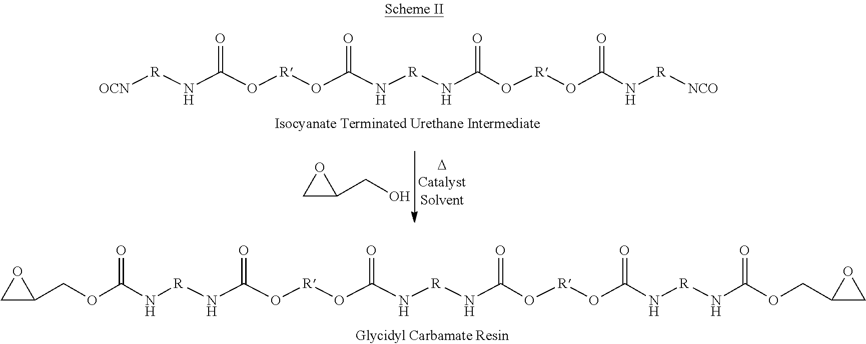

In one embodiment, for the synthesis of linear GC-resins of the invention, the stoichiometric equivalent amount of NCO and glycidol based on total —NCO and —OH groups is 1.0:1.0 (NCO:glycidol). In another embodiment, an excess of glycidol can be used in the reaction, but may be removed prior to using the resin.

In one embodiment, the reaction to make the linear GC-resin may be carried out from about 40° C. to about 90° C., more preferably from about 45° C. to about 55° C. For example, glycidol may be added at about 40° C. and the reaction may then be continued between about 45° C. to about 55° C. The reaction temperature may then be increased in the range of about 60° C. to about 65° C. in the later stage of the reaction until the —NCO peak in the FTIR spectrum disappears completely. In some embodiments, small amounts of glycidol may be added to ensure complete consumption of isocyanate.

### Coating Compositions and Coated Articles

The present invention also relates to coating compositions comprising at least one linear GC-resin of the invention and at least one curing agent. The curing agent serves to crosslink the coating compositions of the invention. The curing agent may be any curing agent known in the art to cure (or crosslink) epoxy resins. The curing agent may be used in the manner and amount known in the art. Suitable curing agents for use in the coating compositions of the invention include those typically employed with epoxy resins, such as aliphatic, araliphatic and aromatic amines, polyamides, amidoamines and epoxy-amine adducts. The coating may be cured at ambient or elevated (e.g., about 80° C.) temperatures. Amine curing agents typically allow the coating to cure at ambient temperatures.

Suitable amine curing agents are those which are soluble in a coating composition of the invention. Amine curing agents known in the art include, for example, diethylenetriamine, triethylenetetramine, tetraethylene-pentamine, etc. as well as 2,2,4- and/or 2,4,4-trimethylhexamethylenediamine; 1,2- and 1,3-diaminopropane; 2,2-dimethylpropylenediamine; 1,4-diaminobutane; 1,6-hexanediamine; 1,7-diaminoheptane; 1,8-diaminooctane; 1,9-diaminononae; 1,12-diaminododecane; 4-azaheptamethylenediamine; N,N″-bis(3-aminopropyl)butane-1,4-diamine; 1-ethyl-1,3-propanediamine; 2,2(4),4-trimethyl-1,6-hexanediamin; bis(3-aminopropyl)piperazine; N-aminoethylpiperazine; N,N-bis(3-aminopropyl)ethylenediamine; 2,4(6)-toluenediamine; dicyandiamine; melamine formaldehyde; tetraethylenepentamine; 3-diethylaminopropylamine; 3,3″-iminobispropylamine; tetraethylenepentamine; 3-diethylaminopropylamine; and 2,2,4- and 2,4,4-trimethylhexamethylenediamine. Exemplary cycloaliphatic amine curing agents include, but are not limited to, 1,2- and 1,3-diaminocyclohexane; 1,4-diamino-2,5-diethylcyclohexane; 1,4-diamino-3,6-diethylcyclohexane; 1,2-diamino-4-ethylcyclohexane; 1,4-diamino-2,5-diethylcyclo-hexane; 1,2-diamino-4-cyclohexylcyclohexane; isophorone-diamine; norbornanediamine; 4,4′-diaminodicyclohexylmethane; 4,4′-diaminodicyclohexylethane; 4,4′-diaminodicyclohexylpropane; 2,2-bis(4-aminocyclohexyl)propane; 3,3′-dimethyl-4,4′-diaminodicyclohexylmethane; 3-amino-1-(4-aminocyclohexyl)propane; 1,3- and 1,4-bis(aminomethyl)cyclohexane; and 1-cyclohexyl-3,4-dimino-cyelohexane. As exemplary araliphatic amines, in particular those amines are employed in which the amino groups are present on the aliphatic radical for example m- and p-xylylenediamine or their hydrogenation products as well as diamide diphenylmethane; diamide diphenylsulfonic acid (amine adduct); 4,4″-methylenedianiline; 2,4-bis(p-aminobenzyl)aniline; diethyltoluenediamine; and m-phenylene diamine. The amine curing agents may be used alone or as mixtures.

Suitable amine-epoxide adducts are, for example, reaction products of diamines such as, for example, ethylenediamine, diethylenetriamine, triethylenetetramine, m-xylylenediamine andior bis(aminomethyl)cyclohexane with terminal epoxides such as, for example, polyglycidyl ethers of polyhydric phenols listed above.

Preferably, amine curing agents used with the coating formulations of the invention are bis(para-aminocyclohexyl)methane (PACM), diethylene triamine (DETA), and 4,4′-methylene dianiline (MDA). Stoichiometry ratios of amine to oxirane of the aqueous coating compositions may be based on amine hydrogen equivalent weight (AHEW) and on weight per epoxide (WPE). A formulation of 1:1 was based on one epoxide reacted with one amine active hydrogen.

In one embodiment, coating compositions according to the invention have an impact resistance of greater than 150, more preferably greater than 160 (as measured by reverse impact (in-lb)). In another embodiment, the coating compositions according to the invention have an impact strength of greater than 50 (as measured by the GE impact test (% area increase)). In another embodiment, the coating compositions according to the invention have an elongation at break of greater than 20 mm.

A coating composition according to the invention may comprise a pigment (organic or inorganic) and/or other additives and fillers known in the art. Such additives or fillers include, but are not limited to, leveling, rheology, and flow control agents such as silicones, fluorocarbons, urethanes, or cellulosics; extenders; reactive coalescing aids such as those described in U.S. Pat. No. 5,349,026; flatting agents; pigment wetting and dispersing agents and surfactants; ultraviolet (UV) absorbers; UV light stabilizers; tinting pigments; extenders; defoaming and antifoaming agents; anti-settling, anti-sag and bodying agents; anti-skinning agents; anti-flooding and anti-floating agents; fungicides and mildewcides; corrosion inhibitors; thickening agents; plasticizers; reactive plasticizers; curing agents; or coalescing agents. Specific examples of such additives can be found in Raw Materials Index, published by the National Paint & Coatings Association, 1500 Rhode Island Avenue, NR, Washington, D.C. 20005.

Examples of flatting agents include, but are not limited to, synthetic silica, available from the Davison Chemical Division of W. R. Grace & Company as SYLOID®; polypropylene, available from Hercules Inc., as HERCOFLAT®; synthetic silicate, available from J. M. Huber Corporation, as ZEOLEX®.

Examples of viscosity, suspension, and flow control agents include, but are not limited to, polyaminoamide phosphate, high molecular weight carboxylic acid salts of polyamine amides, and alkylene amine salts of an unsaturated fatty acid, all available from BYK Chemie U.S.A. as ANTI TERRA®. Further examples include, but are not limited to, polysiloxane copolymers, polyacrylate solution, cellulose esters, hydroxyethyl cellulose, hydroxypropyl cellulose, polyamide wax, polyolefin wax, hydroxypropyl methyl cellulose, polyethylene oxide, and the like.

Another embodiment of the invention relates to a method of preparing a highly flexible coating composition. In one embodiment, this method comprises the step of blending at least one linear GC-resin of the invention with at least one curing agent, preferably an amine curing agent. Suitable amine curing agents are the same as described above for those suitable for the coating compositions of the invention.

In another embodiment of the invention, the invention relates to an article of manufacture comprising a coating composition of the invention. The coating compositions of the invention may be used to form coatings on the following substrates: wood, steel, aluminum, plastic, and glass. The invention also provides methods for coating such substrates by applying the coating composition to the substrate. The coating may be applied by methods know in the art such as drawdown, conventional air-atomized spray, airless spray, roller, brush. The coating may be cured at ambient temperatures or above.

### EXAMPLES

### Materials

Diisocyanates used for the synthesis were hexamethylene diisocyanate (HDI), bis(4-isocyanatocyclohexyl)methane (H₁₂MDI), and trimethyl hexamethylene diisocyanate (TMDI). HDI, H₁₂MDI, and TMDI were Desmodur H, Desmodur W, and Vestanat TMDI, respectively. See Table 1, below. Desmodur H and Desmodur W were obtained from Bayer MaterialScience and Vestanat TMDI was obtained from Evonik. The diols used were 2-butyl-2-ethyl-1,3 propane diol (BEPD) (Aldrich), neopentyl glycol (NPG) (Aldrich), and diethylene glycol (DEG) (Sigma-Aldrich). The triol used to provide some branched oligomers was trimethylol propane (TMP) (Aldrich). Glycidol was supplied by Dixie Chemical. Glycidol was refrigerated to minimize the formation of impurities. Dibutyltindilaurate (DBTDL), purchased from Aldrich, was used to catalyze the isocyanate and hydroxyl reactions to form the glycidyl carbamate (GC) resins. All reagents were used as received without any further purification.

Solvents were used during the resin synthesis to reduce viscosity and facilitate the synthesis reaction. The solvents used were methyl n-amyl ketone (MAK) (Aldrich), ethyl 3-ethoxy propionate (EEP) (Aldrich), and tertiary butyl acetate (TBA) (Ashland). TBA and EEP were also used in coatings formulations. Air Products provided the two amine crosslinkers, para-aminocyclohexyl methane (PACM) and Ancamide-2353 (A-2353), having hydrogen equivalent weights (g/H) of 52.5 and 114, respectively. Ancamide-2353 is a mixture of polyamides of different molecular weights.

Synthesis of Glycidyl Carbamate Functional Resins

As shown in Scheme III, below, the synthesis reaction of the linear GC-resins of the invention was carried out in two steps. In the first step, an isocyanate terminated urethane compound was synthesized using diisocyanates and diols. A small amount of triol was also used in selected resin compositions to introduce some branched oligomers having a higher functionality. In the second step, the linear GC-resin was synthesized by end capping the isocyanate terminated urethane compound with glycidol. A 500 mL four neck reaction vessel was used for the synthesis of the linear GC-resins of the invention. The vessel was fitted with a condenser, nitrogen inlet, Model 210 J-KEM temperature controller, heating mantle, and mechanical stirrer. A water bath was used to maintain the reaction temperature. The molar ratio of isocyanates and hydroxyl groups used for the synthesis of the isocyanate terminated urethane compound was 1.0:0.66. For the synthesis of linear GC-resins, the stoichiometric equivalent amount of NCO and glycidol based on total —NCO and —OH groups was 1.0:1.0 (NCO:glycidol). A series of linear GC-resins of the invention were synthesized using linear aliphatic diisocyanates, and combinations of diols and triol. Table 1, below, shows the compositions of the linear GC-resins synthesized.

During the synthesis of resins, the reaction vessel was charged with the required amounts of diols and optional triol. Solid diols and triol were heated to melt and mixed with solvents before addition of solvent mixture and diisocyanate. The reaction mixture was stirred for about 30 min. to ensure a homogeneous mixture. The catalyst (DBTDL), in the form of solution in TBA (1-2% by wt.), was added after the completion of mixing. The amount of catalyst added was 0.03% by wt. (of the total reaction charge). The reaction was carried out between 65-80° C. until the required value for % NCO (determined by titration) was reached before addition of glycidol. Glycidol was added at 40° C. and reaction was further carried out between 45-55° C. for 3-4 hrs. The reaction temperature was increased in the range of 60-65° C. in the later stage of the reaction until the —NCO peak in the FTIR spectrum disappeared completely. In some cases small amounts of glycidol were added to ensure complete consumption of isocyanate. After the completion of the reactions, the resins were collected in glass jars.

In addition to diisocyanate based GC resins, polyisocyanate based GC resins such as biuret glycidyl carbamate (BGC) synthesized by reacting hexamethylene diisocyanate biuret with glycidol and its modified versions from previous studies were used for comparison of flexibility of coatings. The synthesis of the resins was carried out as described in a previous publication. See Harkal, U D, Muehlberg, A J, Li, J, Garrett, J T, Webster, D C, “The influence of structural modification and composition of glycidyl carbamate resins on their viscosity and coating performance,” J. Coat. Technol. Res. 7(5):531-546 (2010), the disclosure of which is incorporated by reference.

NCO Titration

Isocyanate content (% NCO) of the isocyanate terminated urethane compound was determined using a back titration method according to ASTM D 2572. A required amount (0.5-1.0 gm) of the NCO terminated urethane intermediate dissolved in 50 mL mixture of toluene and isopropyl alcohol (1:1 weight. ratio) was reacted with an excess (25 mL) of 0.1N di-n-butyl amine solution. Unreacted di-n-butyl amine was titrated against 0.1 N HCl using bromophenol blue as an indicator. The end point of the titration was the appearance of a yellow color.

Characterization

FTIR measurements were performed using either a Nicolet Magna-850 or Nicolet 8700 FTIR spectrometer. Sample aliquots were taken and coated on a potassium bromide salt plate. Spectra acquisitions were based on 64 scans with a data spacing of 1.98 cm⁻¹. The change in band absorption of isocyanate (2272 cm⁻¹), —OH and —NH (3750-3000 cm⁻¹), amide (1244 cm⁻¹), and epoxide (910 cm⁻¹) bands were used to follow the reaction progress.

Epoxy equivalent weight of the resins was determined by titration with hydrogen bromide (HBr) according to ASTM D1652. A required amount of resin (0.06 to 0.8 g) was dissolved in 5-10 mL of chloroform and was titrated against a standardized HBr solution prepared in glacial acetic acid. The indicator used was a solution of crystal violet in glacial acetic acid. The end point of the titration was the appearance of a permanent yellow-green color.

Solids content of the resins was determined according to the procedure described in ASTM D 2369. About 1.00 g sample of a resin was weighed in an aluminum boat and dissolved in TBA. The aluminum boats were kept in an oven at 120° C. for one hour. The weights of the sample before and after heating in the oven were used to determine the % solids of the resins.

Coating Preparation

GC coatings were prepared from the GC resins using PACM and Ancamide 2353 crosslinkers. The amine active hydrogen:epoxy equivalent ratio was 1:1 in all of the formulations and based on the determined epoxy equivalent weight of the GC resins. The solvents used for the coating formulations were ethyl 3-ethoxy propionate (EEP) (20% by wt. of total resin) and tertiary butyl acetate (TBA) (20% by wt. of total resin). The formulations were warmed to around 50° C. to 60° C. to ensure homogeneous mixing. The formulations were allowed to sit for 15 min before making drawdowns. The films were applied at a wet film thickness of 6 mils using a drawdown bar on steel panels (smooth finished Q panels, type QD36, 0.5×76×152 mm) cleaned with p-xylene. Films were also applied on Alodine 5700 treated aluminum panels (aluminum alloy-Al2024 T0, 0.032″) to study their impact resistance and adhesion. Alodine 5700 is a non-chromate pretreatment provided by Henkel. Films were applied to glass panels to obtain free films for dynamic mechanical analysis (DMA) and tensile test measurements. The coated panels were kept at ambient conditions overnight and the next day the coated panels were cured in an oven at 80° C. for 1 hr. All the coated panels were kept at ambient conditions for fourteen days after curing before testing. The coatings were used for reverse impact, MEK double rubs, conical mandrel, Konig hardness, cross-hatch adhesion, DSC, and TGA tests.

The free coating films for DMA and tensile test were obtained by immersing the coated glass panels in water overnight and removing the films next day. The free films were tested the following day after drying them at ambient conditions overnight.

The coating samples used for salt spray tests were applied in two coats on steel and treated aluminum panels to minimize local defects in the coatings. The steel panels were wiped with p-xylene for degreasing before applying coatings on them. The aluminum panels used in these experiments were cleaned using MEK for degreasing and Brulin Cleaner (Formula 815MX) with abrasive pad. The aluminum panels were further treated with deoxidizer solution (35% butanol, 25% isopropanol, 18% orthophosphoric acid, and 22% volume deionized water) and Alodine 5700 (chromate free conversion coating). The treated aluminum panels were kept at ambient conditions over night before applying coatings on them. The first coat was drawn down at 4 mils and kept at ambient conditions for two days before curing at 80° C. for 1 hr. The second coat at 5 mils was drawn down on the previously coated and cured panels. After keeping the coatings at ambient conditions for two days, the coatings were again cured at 80° C. for 1 hr. Finally, all the coatings were kept at ambient conditions for 8-9 days before they were placed in the salt spray chamber.

Coating Performance

König pendulum hardness of the coatings was measured following ASTM D 4366. The hardness test results are reported in seconds (sec). Reverse impact strength of the coatings was determined following ASTM D 2794 using a Gardener impact tester. The maximum drop height was 43 inches and the drop weight was 4 pounds. Crazing or loss of adhesion was noted and inch-pounds (in-lbs) were reported at film finish failure. Samples that did not fail were noted as having an impact strength of >172 in-lbs. Flexibility of the coatings was also determined using GE flexibility impact tester according to ASTM D 6905. The conical mandrel test was also used according to ASTM D 522 for the determination of flexibility of the coatings. The results of the flexibility test were reported as the length of a crack (cm) formed on the coating during the test. Methyl ethyl ketone (MEK) double rubs test was used according to ASTM D 5402 to assess the chemical resistance and development of cure. A 26-ounce hammer with three layers of cheesecloth wrapped around the hammerhead was soaked in MEK. The hammer head was rewet with MEK after 30-50 double rubs. Once mar was achieved, a number of double rubs was noted. Cross hatch adhesion of the coatings was evaluated using a Gardco cross hatch adhesion instrument following ASTM D 3359.

Differential Scanning Calorimetry (DSC)

A TA Instruments Q 1000 differential scanning calorimeter (DSC) coupled with an auto sampler accessory was used to determine the glass transition temperature (T_{g}) of the coatings. DSC experiments were performed by placing a sample into conventional aluminum pans. The samples were subjected to a heat-cool-heat cycle. The samples were heated to 200° C. and then cooled to −75° C. and held there for 5 min. DSC thermograms were taken from −75° C. to 250° C. A heating rate of 10° C. min⁻¹ was used during the experiments. Glass transition temperature was determined as the temperature at the mid-point of the inflection in the second DSC cycle.

Thermogravimetric Analysis (TGA)

TGA was performed using a TA Instruments Q 500. Temperature was ramped from ambient to 800° C. with a ramp rate of 10° C. min⁻¹. A nitrogen atmosphere was used during the test. Weight retained was plotted as a function of temperature.

Dynamic Mechanical Analysis (DMA)

A TA Instruments Q 800 Dynamic Mechanical Analysis system was used to determine the viscoelastic properties of the cured coating films. The dimensions of the free films used were of 23 to 26 mm in length, 5 mm in width, and 0.09 to 0.1 mm in thickness. Poisson's ratio was assumed to be 0.4 for all of the coating films. The experiments were carried out within a temperature range of −20° C. to 200° C. with a temperature ramp rate of 5° C. min⁻¹ at a frequency of 1 Hz. The crosslink density of the coatings was calculated from the storage modulus values (well above T_{g}) obtained in DMA experiments. The equation 1 was used to calculate the crosslink density of the coatings (Hill, L W, “Determination of Crosslink Density in Thermoset Coatings,” Polym. Mater. Sci. Eng. 77:387-388 (1997); Skaja, A, Fernando, D, Croll, S, “Mechanical Property Changes and Degradation During Accelerated Weathering of Polyester-Urethane Coatings,” J. Coat. Technol. Res. 3(1):41-51 (2006)):

E′=3vₑRT (1)

where E′ is the storage modulus (Pa), vₑ is the crosslink density (mol/L), R is the gas constant (8.3 J/K/mol), and T is the temperature (K).

Tensile Test

Tensile testing of the coatings was performed using an Instron 5542. The test specimens were prepared according to ASTM D 638-5. The test was carried out at 10 mm/min at ambient conditions. Elongation at break and Young's modulus of the coatings were determined.

Salt Spray Test (ASTM B 117)

The coated panels were scribed and exposed to continuous salt spray (5% NaCl in deionized water) fog at 35° C. for ten days. The images of the coatings were taken periodically by scanning.

### Results

Synthesis of GC Resins Based on Diisocyanates and Diols

The synthesis of the linear GC-resins was carried out as a two step reaction. The first step of the reaction was the synthesis of an isocyanate terminated urethane compound and the second step was end capping of the urethane compound with glycidol. A schematic of the synthesis reaction is shown in Scheme III.

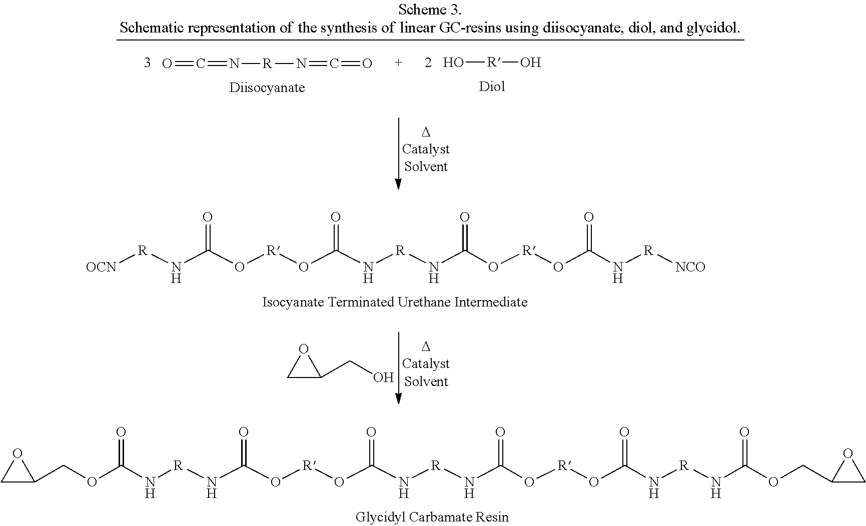

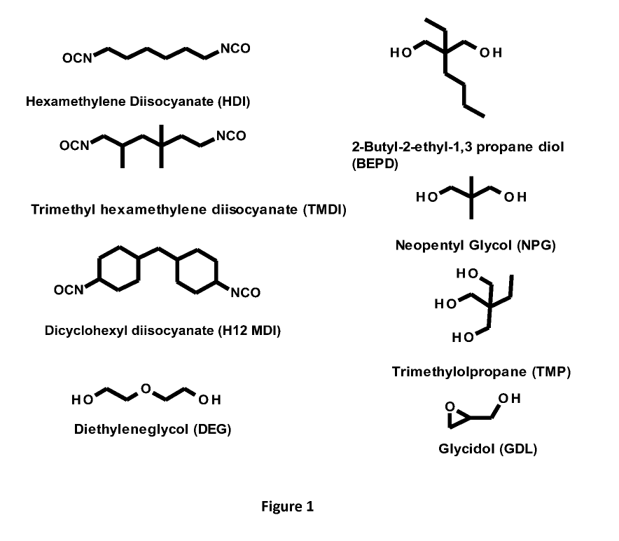

A stoichiometric excess of the diisocyanate was used in the first stage at a ratio to yield an isocyanate-terminated oligomer having an average of five monomer units. Then, the unreacted isocyanate groups were reacted with glycidol to yield the final GC resin. The structures of the raw materials used in the synthesis experiments are shown in FIG. 1. Two aliphatic and one cycloaliphatic diisocyanate were used with all resins containing BEPD. BEPD was selected as the primary diol since its long sidechains can lead to reduced viscosity of the resin. Resins were also synthesized using a combination of BEPD with NPG and DEG to determine their effect on properties. Two resin compositions used a small amount of triol, TMP, to increase the functionality of the resin. Thus, seven GC resins were synthesized based on three diisocyanates and by varying the stoichiometric amount of diols and triol during the synthesis. Table 1 shows the compositions of the GC resins synthesized.

For the comparison of flexibility of coatings based on polyisocyanate based GC resins with that of diisocyanate based GC resins, biuret glycidyl carbamate (BGC) and modified GC resins synthesized in a previous study were used. See Harkal, U D, Muehlberg, A J, Edwards, P A, Webster, D C, “Novel water-dispersible glycidyl carbamate (GC) resins and waterborne amine-cured coatings,” J. Coat. Technol. Res. 8(6):735-747 (2011). The modified GC resins were BGC-EP 15% and BGC-EP 25% where ethyleneglycol propylether (EP) was used as a modifier at 15 and 25 mole % to replace glycidol. The modification of the polyisocyanate based resin was carried out to reduce resin viscosity which also resulted in reduction of epoxy equivalent weight. The viscosity values of BGC, BGC-EP 15%, and BGC-EP 25% were 350×10⁴, 130×10⁴, and 808×10³ mPa·s. Epoxy equivalent weight (theo, g/eq) of BGC, BGC-EP 15%, and BGC-EP 25% were 249, 299, and 343, respectively.

TABLE 1
Recipes for the synthesis and properties of GC resins.

| | Isocyanate terminated urethane intermediate | | | | | End capping by glycidol | | | | Solvents | | | |
|---|---|---|---|---|---|---|---|---|---|---|---|---|---|
| | | | Mole | % NCO | | Weight | Mole | EEW (g/eq.) | | (g) | | | Solids |
| Resin | Composition | Weight (g) | (mol) | Theo. | Act. | (g) | (mol) | Theo. | Act. | TBA | MAK | EEP | (%) |
| R1 | HDI | 150.00 | 0.89 | 10.93 | 10.47 | 42.18 | 0.57 | 458 | 510 | None | 25.0 | None | 90 |
| | BEPD | 47.80 | 0.30 | | | | | | | | | | |
| | NPG | 30.90 | 0.30 | | | | | | | | | | |
| R2 | HDI | 150.00 | 0.89 | 10.75 | 10.39 | 44.96 | 0.61 | 486 | 502 | None | 35.0 | None | 91 |
| | BEPD | 95.37 | 0.59 | | | | | | | | | | |
| R3 | TMDI | 100.17 | 0.48 | 9.39 | 8.67 | 20.92 | 0.28 | 521 | 566 | 15.0 | None | None | 91 |
| | BEPD | 25.54 | 0.16 | | | | | | | | | | |
| | NPG | 16.52 | 0.16 | | | | | | | | | | |
| R4 | HDI | 100.00 | 0.60 | 10.44 | 10.66 | 28.00 | 0.38 | 483 | 450 | 20.0 | None | None | 94 |
| | BEPD | 56.95 | 0.36 | | | | | | | | | | |
| | TMP | 3.55 | 0.03 | | | | | | | | | | |
| R5 | HDI | 100.30 | 0.60 | 11.50 | 9.00 | 22.20 | 0.30 | 446 | 418 | 35.0 | None | None | 95 |
| | DEG | 29.27 | 0.28 | | | | | | | | | | |
| | BEPD | 12.72 | 0.08 | | | | | | | | | | |
| | TMP | 3.54 | 0.03 | | | | | | | | | | |
| R6 | TMDI | 101.6 | 0.48 | 9.33 | 9.00 | 21.05 | 0.28 | 486 | 466 | 15.0 | None | None | 94 |
| | DEG | 23.40 | 0.22 | | | | | | | | | | |
| | BEPD | 10.05 | 0.06 | | | | | | | | | | |
| | TMP | 2.82 | 0.02 | | | | | | | | | | |
| R7 | H₁₂MDI | 150.00 | 0.57 | 7.82 | 7.32 | 27.10 | 0.37 | 627 | 681 | 30.0 | 20.0 | 10.0 | 82 |
| | BEPD | 60.26 | 0.38 | | | | | | | | | | |

Initial Screening of Coating Based on Impact and Solvent Resistance

The first phase of this research involved screening of the resins in coatings to identify those coatings having a combination of good flexibility and solvent resistance. For the screening study, PACM crosslinked GC coatings were prepared using GC resins R1 to R6, BGC, and modified GC resins (BGC-EP 15% and BGC-EP 25%). The GC coatings prepared from polyisocyanate based GC resins (BGC and modified GC resins) were used to compare their flexibility with the coatings prepared from diisocyanate based GC resins. (Resin R7 was prepared in a second phase of the study and coatings based on resin R7 were characterized along with the screened coatings.) For the initial screening, coatings were prepared on aluminum and steel substrates. Reverse impact test was carried out on the coatings prepared on aluminum substrate. MEK double rubs test was carried out on the coatings prepared on steel substrates. Table 2 shows the results of reverse impact and MEK double rubs tests.

TABLE 2
Performance of PACM crosslinked GC coatings in screening study

| GC Coatings | Reverse Impact (in-lb) | MEK Double Rubs |
|---|---|---|
| BGC | 28 | >400 |
| BGC-EP 15% | 96 | >400 |
| BGC-EP 25% | 132 | >400 |
| R1 | >172 | >400 |
| R2 | >172 | 325 |
| R3 | >172 | 50 |
| R4 | >172 | 100 |
| R5 | >172 | 80 |
| R6 | >172 | 46 |

The initial screening showed that BGC and modified GC coatings had relatively low impact resistance compared to the GC coatings obtained from the linear diisocyanate, diols, and triol based GC resins of the invention. Solvent resistance of BGC, modified GC, R1, and R2 coatings was higher compared to that of the other GC coatings. Coatings obtained from resins R1 and R2 showed high impact strength and high solvent resistance. The compositions of HDI, BEPD, and NPG in resin R1 and HDI and BEPD in resin R2 produced coatings with a combination of both high impact strength and high solvent resistance. Resins based on DEG and TMDI had good impact resistance, but generally poor solvent resistance. The coatings obtained from resins R4 and R5 had high impact strength but low solvent resistance. Addition of higher functionality through TMP in resins R4, R5, and R6 did not result in improved solvent resistance. Thus, based on the initial screening, coatings based on R1 and R2 resins were selected for further characterization.

Further Analysis of Properties of the Screened Coatings

Six GC coatings were prepared from the screened GC resins, R1, R2, and a third resin, R7, in combination with two amine crosslinkers. Resin R7 was synthesized for the second phase of the study to examine the influence of the more rigid cycloaliphatic structure of H₁₂MDI on the coatings properties. The crosslinkers used were PACM and a polyamide resin, Ancamide 2353. PACM crosslinked GC coatings from resins R1, R2, and R7 were labeled F1, F2, and F3, respectively. Ancamide 2353 crosslinked coatings from resins R1, R2, and R7 were labeled L1, L2, and L3, respectively. Table 3 shows the GC coatings properties such as crosslink density, adhesion, flexibility, solvent resistance, hardness, glass transition (T_{g}) temperature, elongation at break, and Young's modulus.

The coatings had high hardness, good adhesion, and high chemical resistance. The flexibility of coatings F1, F2, L1, and L2 was higher compared to that of the coatings F3 and L3 as indicated by their highest impact strength, 60 percent area increase in the GE impact test, no crack in conical mandrel test, and high elongation at break in tensile test. F3 and L3 coatings are obtained from GC resin R7 composed of cycloaliphatic diisocyanate, H₁₂MDI, whereas the other GC coatings F1, F2, L1, and L2 were obtained from resins R1 and R2 composed of aliphatic diisocyanate, HDI. The cycloaliphatic structure of H₁₂MDI is considered highly rigid and responsible for very low flexibility compared to the aliphatic diisocyanate, HDI. See, e.g., Yilgor, I, Yilgor, E, “Structure-Morphology-Property Behavior of Segmented Thermoplastic Polyurethanes and Polyureas Prepared without Chain Extenders,” Polym. Rev. (Philadelphia, Pa., U.S.) 47(4):487-510 (2007); Dearth, R S, Mertes, H, Jacobs, P J, “An overview of the structure/property relationship of coatings based on 4,4′-dicyclohexylmethane diisocyanate (H12MDI),” Prog. Org. Coat. 29(1-4):73-79 (1996); Yoo, H-J, Lee, Y-H, Kwon, J-Y, Kim, H-D, “Comparison of the properties of UV-cured polyurethane acrylates containing different diisocyanates and low molecular weight diols,” Fibers Polym. 2(3):122-128 (2001); Adhikari, R, Gunatillake, P A, Meijs, G F, McCarthy, S J, “The effect of diisocyanate isomer composition on properties and morphology of polyurethanes based on 4,4′-dicyclohexyl methane diisocyanate and mixed macrodiols (PDMS-PHMO),” J. Appl. Polym. Sci. 73(4):573-582 (1999). Thus, the high flexibility of coatings F1, F2, L1, and L2 obtained from resins R1 and R2 can be attributed to the resin composition containing the linear aliphatic diisocyanate (HDI). The influence of rigid H₁₂MDI was also reflected in higher hardness values for coatings F3 and L3 compared to that of the other coatings. While coatings F3 and L3 had lower crosslink density compared to that of the others, due to the higher equivalent weight of the resin, their glass transition temperature was higher than the others due to the rigid cycloaliphatic H₁₂MDI.

The influence of the type of amine crosslinker was prominent on solvent resistance (MEK double rubs) and hardness. PACM crosslinked GC coatings had higher hardness and solvent resistance compared to that of the Ancamide 2353 crosslinked coatings.

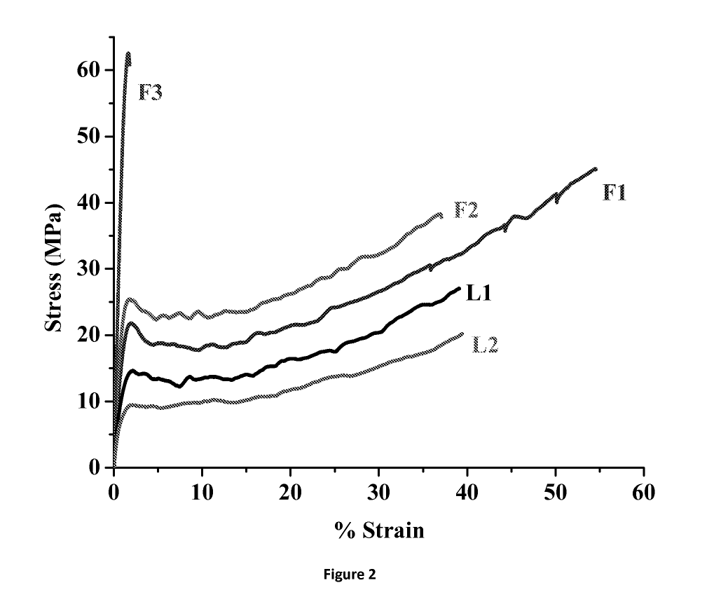

FIG. 2 shows stress vs. strain plots for F1, F2, F3, L1, and L2 coatings. The highly brittle nature of coating L3 did not produce intact samples suitable for tensile testing. F1 coating based on resin R1 composed of HDI, BEPD and NPG, and crosslinked with PACM exhibited the highest elongation at break. However, the similar composition in F2 except no NPG showed lower elongation at break compared to that of F1.

TABLE 3
Properties of GC coatings.

| | | | | | | | | | | |
|---|---|---|---|---|---|---|---|---|---|---|
| | | GE impact | | | | | | | Köning | T_{g} |
| | Reverse | test | Crosslink | Conical | Elongation | Young's | Crosshatch | MEK | pendulum | DSC first |
| GC | impact | (% area | density | mandrel (cm) | at break | modulus | adhesion* | double | hardness | dry cycle |
| coating | (in-lb)* | increase) | (mol/L) | (0 cm = best) | (mm) | (Mpa) | (5B = Best) | runs | (sec) | (° C.) |

PACM crosslinked

| | | | | | | | | | | |
|---|---|---|---|---|---|---|---|---|---|---|
| F1 | >172 | 60 | 0.660 | 0 | 34 | 2600 | 5B | >400 | 156 | 25 |
| F2 | >172 | 60 | 0.575 | 0 | 23 | 3176 | 5B | >400 | 153 | 25 |
| F3 | 14 | <10 | 0.172 | 1 | 1 | 5400 | 5B | >400 | 223 | 55 |
| | | | | | | | | | | endotherm |

Ancamide 2353 crosslinked

| | | | | | | | | | | |
|---|---|---|---|---|---|---|---|---|---|---|
| L1 | >172 | 60 | 0.549 | 0 | 22 | 2000 | 5B | 190 | 100 | 20 |
| L2 | >172 | 60 | 0.394 | 0 | 26 | 1445 | 5B | 170 | 81 | 18 |
| L3 | <8 | <10 | 0.259 | 1 | Films too | Films too | 5B | 300 | 208 | 57 |
| | | | | | brittle for | brittle for | | | | endotherm |
| | | | | | sampling | sampling | | | | |

*Tests performed on aluminum substrate (Al024-T0)

### Dynamic Mechanical Analysis

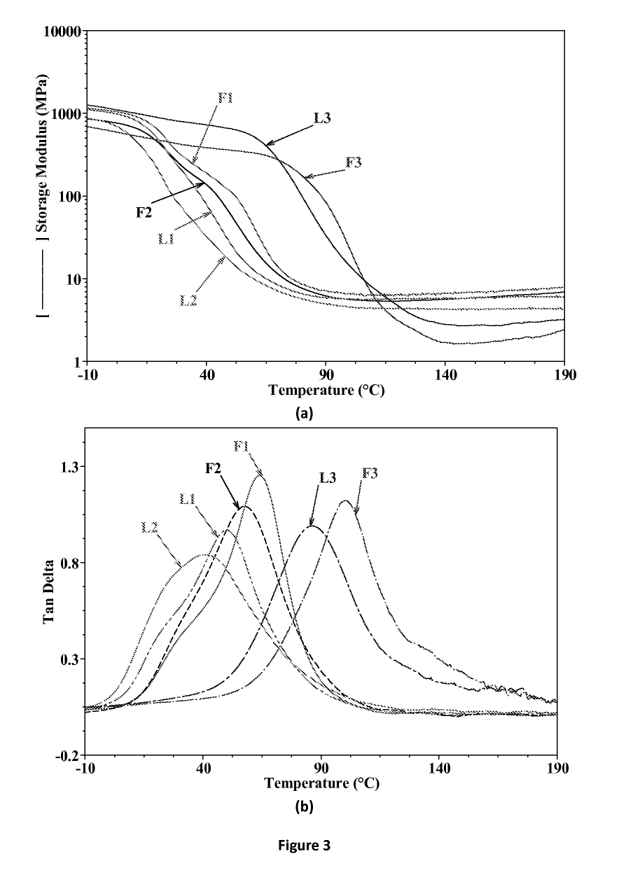

Dynamic mechanical analysis was used to further understand the influence of the composition of GC resins and type of amine crosslinker on the coating properties. FIG. 3 shows (a) storage modulus and (b) tan δ curves for the GC coatings. Storage modulus of coatings indicates the stiffness (rigidity) of coatings and it decreases in the transition region making coatings more flexible. See Menczel, J D, Prime, R B, Thermal Analysis of Polymers Fundamentals and Applications, John Wiley & Sons, Inc., New Jersey (2009).

Transition temperatures for F1, F2, L1, and L2 coatings were much lower than that for F3 and L3 coatings. This indicated lower stiffness and higher flexibility of F1, F2, L1, and L2 coatings compared to that of F3 and L3 coatings. F1, F2, L1, and L2 coatings are obtained from GC resins (R1 and R2) based on the flexible aliphatic diisocyanate (HDI). On the other hand, F3 and L3 coatings are obtained from GC resins based on the rigid cycloaliphatic diisocyanate (H₁₂MDI). The transition region and tan δ curves for H₁₂MDI based GC coatings (F3 and L3) are well above room temperature. High flexibility of HDI based GC coatings (F1, F2, L1, and L2) can be correlated to the appearance of their transition region near room temperature. The composition of the crosslinker influenced the breadth of the tan δ curves. For all of the GC coatings, Ancamide 2353 crosslinked coatings showed broader tan δ peaks compared to the corresponding PACM crosslinked GC coatings. The broadening of tan δ peaks indicates non-uniformity in the crosslinked network. See Higginbottom, H P, Bowers, G R, Ferrell, P E, “Cure of Secondary Carbamate Groups by Melamine-Formaldehyde Resins,” J. Coat. Technol. 71(894):49-60 (1999). Also, a comparison of the tan δ peaks for the H₁₂MDI based coatings (F3 and L3) with that of the other coatings shows that F3 and L3 coatings have relatively more symmetric and narrow tan δ peaks compared to that of the other coatings. Thus, the H₁₂MDI based L3 coating had a more uniform crosslinked network compared to that of the HDI based L1 and L2 coatings.

Thermal Stability

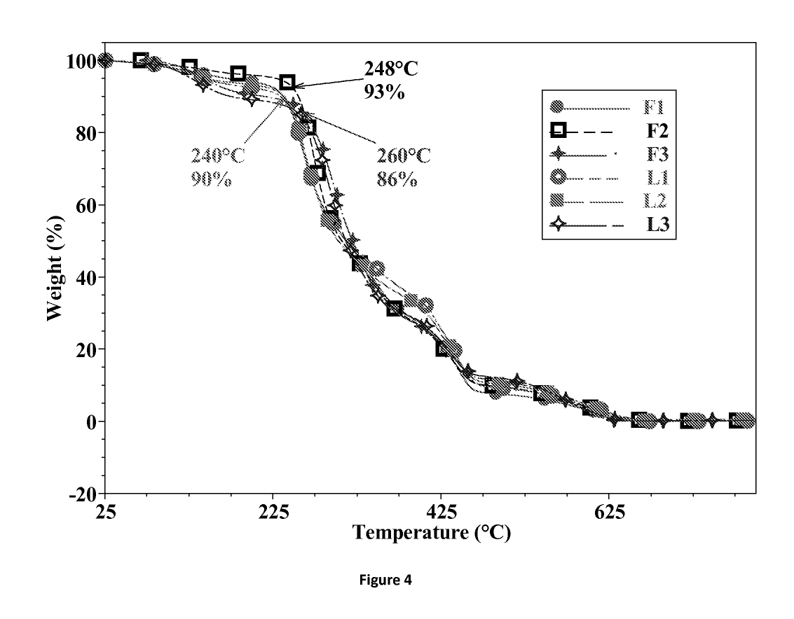

The thermal stability of the amine crosslinked GC coatings was studied using TGA. TGA plots for the GC coatings are shown in FIG. 4. The GC coatings show stability around 125° C. The onset temperature for thermal degradation was found to be between 240 and 260° C. and weight loss in this temperature range was between 7 to 16%.

Salt spray Test

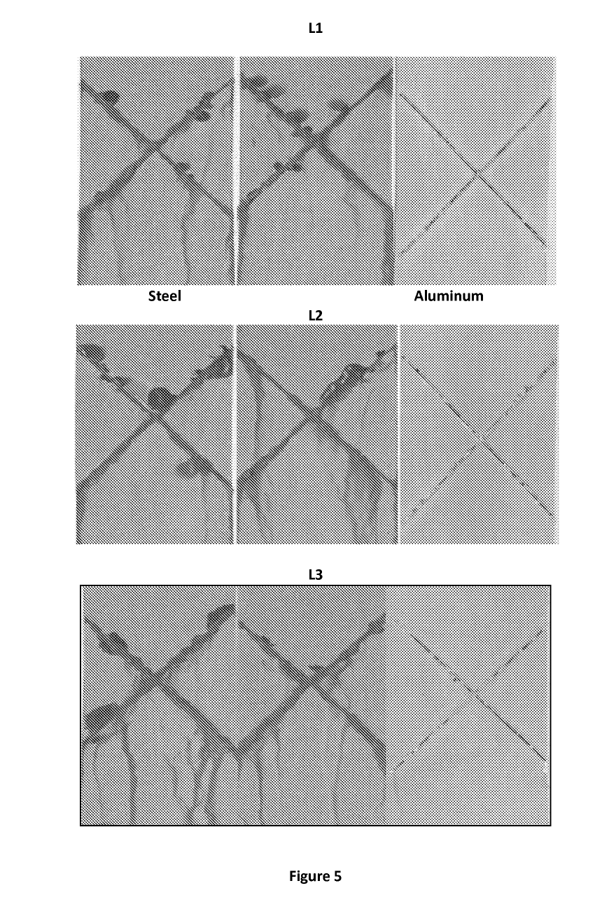

ASTM B 117 Salt spray test is the oldest standard test used to compare the corrosion resistance of coatings. The salt spray test was performed on the coatings on scribed steel and aluminum panels. The scribe on the coatings and panels results in physical damage to the coatings and the underlying substrate. Performance of the coatings on steel and aluminum panels was studied under continuous exposure to the salt fog (5% NaCl solution) over 240 hrs. FIG. 5 shows images of coatings L1, L2, and L3, two of each on steel panels and one of each on an aluminum panel after 240 hrs of salt spray test.

After 240 hrs of salt spray, coating L1 on one steel panel showed under film corrosion while the coating on the second steel panel showed blister formation. Coatings L2 on both the steel panels had blisters after 240 hrs of salt spray. Coating L3 on one of the steel panels did not show any blister formation, film delamination, or under-film corrosion. Coating L3 on the other steel panel showed under film corrosion but no blisters.

All the coatings on aluminum panels were intact after 240 hrs of salt spray with no signs of blistering, delamination, or creep. Aluminum panels had been pretreated with Alodine 5700. The Alodine treatment improved the adhesion and corrosion performance of the coatings on aluminum substrate. See, e.g., Zhai, Y, Zhao, Z, Frankel, G S, Zimmerman, J, Bryden, T, Fristad, W, “Surface pretreatment based on dilute hexafluorozirconic acid,” Proceedings of the Tri-Service Corrosion Conference 1-16 (2007); Smith, P, Miller, C, “Assessing the performance of chromate-free pretreatment options for CARC systems. Comprehensive research efforts aimed at prevention and early detection of corrosion in U.S. Military equipment,” Metal Finishing 105(9):62-70 (2007).

### CONCLUSIONS

GC resins having a more linear structure are feasible as highly flexible coatings and the composition of the GC resins influenced the coating performance. Coatings based on GC resins R1 and R2 with monomers HDI, BEPD, and NPG, and HDI and BEPD, respectively, had a good combination of properties such as high flexibility and high solvent resistance. The coatings obtained from the resin R1 with monomers HDI, BEPD, and NPG had the highest flexibility. The composition of HDI and TMDI based resins with NPG showed good flexibility, however, TMDI based GC coatings showed reduced solvent resistance. A small amount of TMP in the resin composition did not show a significant influence on solvent resistance. Coatings based on GC resin R7 with a composition of H₁₂MDI and BEPD had the least flexibility, the highest modulus and T_{g}, and had high solvent resistance, and high corrosion resistance compared the other coatings characterized in this research work. Thus, the structure of diisocyanate and diol influenced the flexibility and T_{g} of the coatings. Linear HDI based coatings had higher flexibility and lower T_{g} than the H₁₂MDI based coatings. The resin compositions with NPG had improved flexibility.

Coating properties such as solvent resistance, hardness, and T_{g} were also influenced by the type of crosslinker used. PACM crosslinked coatings exhibited higher solvent resistance, hardness, and T_{g} compared to that of the A-2353 crosslinked coatings.

Salt spray testing showed that the coatings based on H₁₂MDI had better corrosion resistance than the HDI based coatings. The substrate treatment also had an influence on the corrosion performance. The coatings on Alodine treated aluminum had better corrosion performance in salt spray test compared to that of the coatings on untreated steel substrate. The coatings on the treated aluminum substrate did not show any blisters or delamination.

1. A linear isocyanate terminated urethane compound, comprising the reaction product of at least one diisocyanate, at least one diol and, optionally, at least one triol,
wherein the diisocyanate has the following structure:
O═C═N—R—N═C═O
wherein R is a divalent hydrocarbyl group selected from a straight or branched C₂-C₁₈ alkylene group, C₂-C₁₈ alkenylene group, and C₂-C₁₈ alkynylene group, and a divalent cyclic group;
wherein the diol has the following structure:
HO—R′—OH
wherein R′ is selected from a divalent hydrocarbyl group and a divalent ether group; and
wherein the optional triol is selected from a C₃-C₁₀ alkyl triol.

2. The compound of claim 1, wherein R is selected from a C₁-C₁₅ alkyl and a C₃-C₁₅ cycloalkyl.

3. The compound of claim 2, wherein R is hexamethylene group or a divalent substituent selected from the group consisting of:

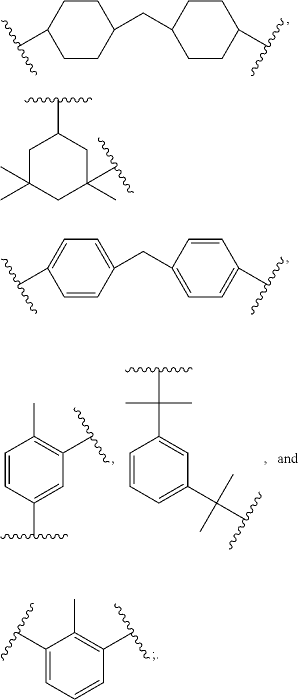

4. The compound of claim 1, wherein the diisocyanate compound is selected from hexamethylene diisocyanate, trimethyl hexamethylene diisocyanate, dicyclohexyl diisocyanate, isophorone diisocyanate, 4,4′-methylene diphenyl diisocyanate, 2,2′-methylene diphenyl diisocyanate, 2,4′-methylene diphenyl diisocyanate, 1,3-phenylene diisocyanate, 1,4-phenylene diisocyanate, 1,4-cyclohexyl diisocyanate, meta-tetramethylxylylene diisocyanate, 2,4-toluene diisocyanate, 2,6-toluene diisocyanate, and mixtures thereof.

5. The compound of claim 1, wherein the diol is selected from diethyleneglycol, 2-butyl-2-ethyl-1,3-propane diol, ethylene glycol, 1,2-propane diol, 1,3-propane diol, 2-methyl-1,3-propane diol, 1,4-butanediol, 2,3-butanediol, 1,5-pentanediol, 1,6-hexanediol, 1,4-cyclohexanedimethanol, neopentyl glycol, and mixtures thereof.

6. The compound of claim 1, wherein the triol is selected from trimethylolpropane, trimethylol ethane, glycerol, and mixtures thereof.

7. The compound of claim 1, wherein the isocyanate terminated urethane compound has the following structure: 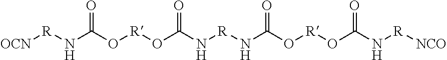
wherein R is a divalent hydrocarbyl group selected from a straight or branched C₂-C₁₈ alkylene group, C₂-C₁₈ alkenylene group, and C₂-C₁₈ alkynylene group, and a divalent cyclic group; and
wherein R′ is selected from a divalent hydrocarbyl group and a divalent ether group.

8. A linear glycidyl carbamate resin, comprising the reaction product of an isocyanate terminated urethane compound and glycidol, wherein the isocyanate terminated urethane compound comprises the reaction product of at least one diisocyanate compound, at least one diol compound, and, optionally, at least one triol compound.

9. The linear glycidyl carbamate resin of claim 8, wherein the linear glycidyl carbamate resin has the following structure: 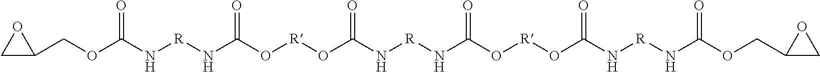
wherein R is a divalent hydrocarbyl group selected from a straight or branched C₂-C₁₈ alkylene group, C₂-C₁₈ alkenylene group, and C₂-C₁₈ alkynylene group, and a divalent cyclic group; and
wherein R′ is selected from a divalent hydrocarbyl group and a divalent ether group.

10. A coating composition comprising:
a) at least one linear glycidyl carbamate resin, and
b) at least one curing agent.

11. The coating composition of claim 10, wherein said at least one curing agent is selected from an amine curing agent.

12. The coating composition of claim 11, wherein said amine curing agent is selected from bis(para-aminocyclohexyl)methane, diethylene triamine, and 4,4′-methylene dianiline.

13. The coating composition of claim 10, wherein said coating composition has an impact resistance of greater than 150 (as measured by reverse impact (in-lb)).

14. The coating composition of claim 10, wherein said coating composition has an impact strength of greater than 50 (as measured by the GE impact test (% area increase)).

15. The coating composition of claim 10, wherein said coating composition has an elongation at break of greater than 20 mm.

16. A method for making an isocyanate terminated urethane compound comprising:
a) reacting at least one diisocyanate compound;
b) at least one diol compound; and
c) optionally, at least one triol compound.

17. The method of claim 16, wherein said diisocyanate compound is selected from hexamethylene diisocyanate, trimethyl hexamethylene diisocyanate, dicyclohexyl diisocyanate, isophorone diisocyanate, 4,4′-methylene diphenyl diisocyanate, 2,2′-methylene diphenyl diisocyanate, 2,4′-methylene diphenyl diisocyanate, 1,3-phenylene diisocyanate, 1,4-phenylene diisocyanate, 1,4-cyclohexyl diisocyanate, meta-tetramethylxylylene diisocyanate, 2,4-toluene diisocyanate, 2,6-toluene diisocyanate, and mixtures thereof.

18. The method of claim 16, wherein said at least one diol is selected from diethyleneglycol, 2-butyl-2-ethyl-1,3-propane diol, ethylene glycol, 1,2-propane diol, 1,3-propane diol, 2-methyl-1,3-propane diol, 1,4-butanediol, 2,3-butanediol, 1,5-pentanediol, 1,6-hexanediol, 1,4-cyclohexanedimethanol, neopentyl glycol, and mixtures thereof.

19. The method of claim 16, wherein said at least one triol is trimethylolpropane.

20. The method of claim 16, wherein said diisocyanate compound is present in a stoichiometric excess relative to the at least one diol and at least one optional triol compound.

21. A method for making a linear glycidyl carbamate resin comprising:
a) reacting an isocyanate terminated urethane compound; and
b) glycidol.

22. The method of claim 21, wherein said isocyanate terminated urethane compound and glycidol are present in a stoichiometrically equivalent amount of NCO and glycidol based on total —NCO and —OH groups.

23. The method of claim 16, further comprising a solvent.

24. The method of claim 23, wherein the solvent is selected from t-butyl acetate, methyl n-amyl ketone, and ethyl 3-ethoxyproprionate.

25. The method of claim 23, wherein the solvent is present in an amount ranging from about 0.1% to about 50.0% by weight of the total reaction mixture.

26. The method of claim 16, further comprising a catalyst.

27. The method of claim 26, wherein the catalyst is selected from dibutyltindilaurate.

28. The method of claim 26, wherein the catalyst is present in an amount ranging from about 0.01% to about 0.1% by weight of the total reaction mixture.

29. A method for making a coating, comprising curing at least one linear glycidyl carbamate resin with at least one curing agent.

30. An article coated with a coating composition comprising at least one linear glycidyl carbamate resin, and at least one curing agent,
wherein said linear glycidyl carbamate resin comprises the reaction product of an isocyanate terminated urethane compound and glycidol,
wherein said isocyanate terminated urethane compound comprises the reaction product of at least one diisocyanate compound, at least one diol compound, and, optionally, at least one triol compound.
